(19) United States
(12) Patent Application Publication
(10) Pub. No.: US 2025/0352180 A1
(43) Pub. Date: Nov. 20, 2025

(54) ULTRASOUND DIAGNOSIS SUPPORT APPARATUS AND ULTRASOUND DIAGNOSIS SUPPORT PROGRAM

(71) Applicant: FUJIFILM Corporation, Tokyo (JP)

(72) Inventor: Natsumi DANNO, Tokyo (JP)

(73) Assignee: FUJIFILM Corporation, Tokyo (JP)

(21) Appl. No.: 19/201,313

(22) Filed: May 7, 2025

(30) Foreign Application Priority Data
May 15, 2024 (JP) 2024-079322

(51) Int. Cl. A61B 8/00; A61B 8/13

(52) U.S. Cl. CPC A61B 8/463; A61B 8/13; A61B 8/483; A61B 8/488; A61B 8/54

(57) ABSTRACT

A communication interface receives a three-dimensional model of a target tissue, which is formed based on medical volume data of a subject, from a medical image analysis server. A cross section conversion unit converts an ultrasonic wave transmission and reception plane in a real space into a model space cross section in a model space of the three-dimensional model, based on position and orientation information of an ultrasound probe acquired by a position and orientation sensor. A model cross sectional image forming unit specifies a target tissue region, which is a region occupied by the target tissue in the model space cross section, by cutting out the three-dimensional model along the model space cross section corresponding to a current position and orientation of the ultrasound probe. A display control unit displays a real-time ultrasound tomographic image and the target tissue region superimposed on the real-time ultrasound tomographic image on a display.

### CROSS REFERENCE TO RELATED APPLICATION

This application claims priority to Japanese Patent Application No. 2024-079322 filed on May 15, 2024, the entire contents of which are incorporated herein by reference.

### BACKGROUND OF THE INVENTION

### 1. Field of the Invention

The present specification discloses improvements in an ultrasound diagnosis support apparatus and an ultrasound diagnosis support program.

### 2. Description of the Related Art

An ultrasound diagnostic apparatus that transmits ultrasonic waves from an ultrasound probe in contact with a body surface of a subject toward the subject, receives reflected waves from the subject at the ultrasound probe, and forms an ultrasound tomographic image of the subject based on reception signals formed from the reflected waves is known.

Conventionally, various techniques have been proposed to form an appropriate ultrasound tomographic image. For example, JP2022-164416A discloses an ultrasonic treatment diagnostic system that accumulates and stores, in a memory, a set of an ultrasound tomographic image, an organ mask indicating an organ region included in the ultrasound tomographic image, and an acoustic shadow mask indicating an acoustic shadow region included in the ultrasound tomographic image, that searches for a reference image in the memory based on a similarity between an organ mask corresponding to an ultrasound tomographic image to be complemented and the stored organ mask, and a similarity between an acoustic shadow mask corresponding to the ultrasound tomographic image to be complemented and the stored acoustic shadow mask, and that complements an acoustic shadow region of the ultrasound tomographic image to be complemented using the reference image.

### SUMMARY OF THE INVENTION

Meanwhile, in the ultrasound tomographic image, it is sometimes not easy to appropriately depict a targeted tissue in the subject (referred to as a “target tissue” in the present specification). For example, in a case where the target tissue is located deep in a gastrointestinal tract as viewed from the body surface, gas in the gastrointestinal tract may obstruct the transmission and reception of the ultrasonic waves, thereby making it difficult to depict the target tissue in the ultrasound tomographic image. In addition, for example, in a case where an examiner (such as a doctor or an ultrasound technician) is not familiar with using the ultrasound diagnostic apparatus, the examiner may not be able to understand how to adjust a position and orientation of the ultrasound probe to suitably depict the target tissue in the ultrasound tomographic image.

An object of an ultrasound diagnosis support apparatus disclosed in the present specification is to support appropriate depiction of a target tissue in an ultrasound tomographic image.

An ultrasound diagnosis support apparatus disclosed in the present specification is an ultrasound diagnosis support apparatus comprising: a three-dimensional model acquisition unit that acquires a three-dimensional model of a target tissue, which is formed based on medical volume data acquired by a medical device; a cross section conversion unit that converts an ultrasound scanning plane including the target tissue into a model space cross section, which is a cross section in a model space of the three-dimensional model, based on position and orientation information indicating a current position and orientation of an ultrasound probe that performs ultrasound scanning in the ultrasound scanning plane; a target tissue region specifying unit that specifies a target tissue region, which is a region occupied by the target tissue in the ultrasound scanning plane, by cutting out the three-dimensional model along the model space cross section; and a display control unit that displays an ultrasound tomographic image, which is formed based on a reception signal obtained by transmitting and receiving an ultrasonic wave to and from the target tissue at the current position and orientation of the ultrasound probe, on a display unit and that displays the target tissue region in a superimposed manner on the ultrasound tomographic image.

The display control unit may change a display aspect of the target tissue region in response to an instruction from an examiner.

In a case where there are a plurality of the target tissues and the target tissue region specifying unit specifies the plurality of target tissue regions, the display control unit may display the target tissue region selected by an examiner and may not display the target tissue region not selected by the examiner.

In a case where there are a plurality of the target tissues and the target tissue region specifying unit specifies the plurality of target tissue regions, the display control unit may display each of the target tissue regions in a different aspect.

The three-dimensional model may have attribute information regarding the target tissue, and the display control unit may display the attribute information of the target tissue corresponding to the target tissue region on the display unit in an aspect associated with the target tissue region.

The target tissue region specifying unit may specify a registration target tissue region, which is a region occupied by the target tissue in a predetermined cross section, by cutting out the three-dimensional model along the predetermined cross section, and the display control unit may display, on the display unit, a medical tomographic image, which is formed by cutting out and reconstructing the medical volume data along the predetermined cross section, and the ultrasound tomographic image and further display the registration target tissue region in a superimposed manner on the ultrasound tomographic image, before conversion processing is performed by the cross section conversion unit.

A color Doppler processing unit that forms a color Doppler image based on the specified target tissue region may be further provided.

The color Doppler processing unit may set a color Doppler region of interest based on the target tissue region.

The color Doppler processing unit may set a color Doppler velocity range based on the target tissue corresponding to the target tissue region.

An image quality adjustment unit that adjusts an image quality of the ultrasound tomographic image based on the target tissue corresponding to the specified target tissue region may be further provided.

In addition, an ultrasound diagnosis support program disclosed in the present specification is an ultrasound diagnosis support program for causing a computer to function as: a three-dimensional model acquisition unit that acquires a three-dimensional model of a target tissue, which is formed based on medical volume data acquired by a medical device; a cross section conversion unit that converts an ultrasound scanning plane including the target tissue into a model space cross section, which is a cross section in a model space of the three-dimensional model, based on position and orientation information indicating a current position and orientation of an ultrasound probe that performs ultrasound scanning in the ultrasound scanning plane; a target tissue region specifying unit that specifies a target tissue region, which is a region occupied by the target tissue in the ultrasound scanning plane, by cutting out the three-dimensional model along the model space cross section; and a display control unit that displays an ultrasound tomographic image, which is formed based on a reception signal obtained by transmitting and receiving an ultrasonic wave to and from the target tissue at the current position and orientation of the ultrasound probe, on a display unit and that displays the target tissue region in a superimposed manner on the ultrasound tomographic image.

With the ultrasound diagnosis support apparatus disclosed in the present specification, it is possible to support appropriate depiction of a target tissue in an ultrasound tomographic image.

### BRIEF DESCRIPTION OF THE DRAWINGS

FIG. 1 is a schematic diagram of a configuration of an ultrasound diagnosis support system according to the present embodiment.

FIG. 2 is a diagram showing an example of a three-dimensional model of a target tissue.

FIG. 3 is a schematic diagram of a configuration of an ultrasound diagnostic apparatus according to the present embodiment.

FIG. 4 is a diagram showing a model space cross section.

FIG. 5 is a diagram showing an example of a model cross sectional image.

FIG. 6 is a diagram showing a first display example of a real-time ultrasound tomographic image and a target tissue region.

FIG. 7 is a diagram showing a second display example of the real-time ultrasound tomographic image and the target tissue region.

FIG. 8 is a diagram showing an example of a display tissue selection screen.

FIG. 9 is a diagram showing a third display example of the real-time ultrasound tomographic image and the target tissue region.

FIG. 10 is a diagram showing a display example of attribute information of the target tissue.

FIG. 11 is a diagram showing an example of a color Doppler region of interest set based on the target tissue region.

FIG. 12 is a diagram showing a display example of a color Doppler image.

FIG. 13 is a flowchart showing a flow of processing of the ultrasound diagnostic apparatus according to the present embodiment.

### DESCRIPTION OF THE PREFERRED EMBODIMENTS

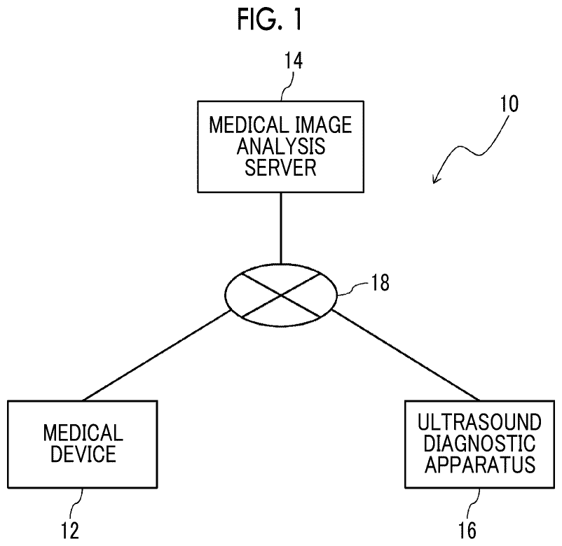

FIG. 1 is a schematic diagram of a configuration of an ultrasound diagnosis support system 10 according to the present embodiment. The ultrasound diagnosis support system 10 includes one or a plurality of medical devices 12, a medical image analysis server 14, and an ultrasound diagnostic apparatus 16 as an ultrasound diagnosis support apparatus. The medical device 12, the medical image analysis server 14, and the ultrasound diagnostic apparatus 16 are connected to each other via a communication line 18, such as a wide area network (WAN) or a local area network (LAN), to be communicable with each other.

The medical device 12 is a device that forms medical volume data of a subject, in particular, medical volume data including a target tissue. The medical device 12 is, for example, a computed tomography (CT) apparatus, a magnetic resonance imaging (MRI) apparatus, an ultrasound diagnostic apparatus, or the like. For example, in a case where the medical device 12 is a CT apparatus, the CT apparatus forms CT data as the medical volume data, in a case where the medical device 12 is an MRI apparatus, the MRI apparatus forms MRI data as the medical volume data, and in a case where the medical device 12 is an ultrasound diagnostic apparatus, the ultrasound diagnostic apparatus forms ultrasound volume data as the medical volume data.

The medical volume data is data in which voxels, each having data, are arranged in three dimensions. The medical volume data has position information (coordinates) in a data space of the medical volume data, which indicates a position of each voxel.

The medical device 12 transmits the formed medical volume data to the medical image analysis server 14.

The medical device 12 forms the medical volume data for the subject and transmits the medical volume data to the medical image analysis server 14, prior to the transmission and reception of ultrasonic waves with respect to the subject by the ultrasound diagnostic apparatus 16 (in other words, the formation of an ultrasound tomographic image). That is, the medical volume data represents the subject (specifically, the target tissue) at a (past) point in time before the (real-time) ultrasound tomographic image is formed by the ultrasound diagnostic apparatus 16.

The medical image analysis server 14 is configured using, for example, a server computer or the like. The medical image analysis server 14 may be implemented through the collaboration of a plurality of server computers. In the present embodiment, the medical image analysis server 14 forms a three-dimensional model of the target tissue based on the medical volume data received from the medical device 12. In the present embodiment, the medical image analysis server 14 forms the three-dimensional model for each of a plurality of target tissues with all tissues included in the medical volume data as the target tissues.

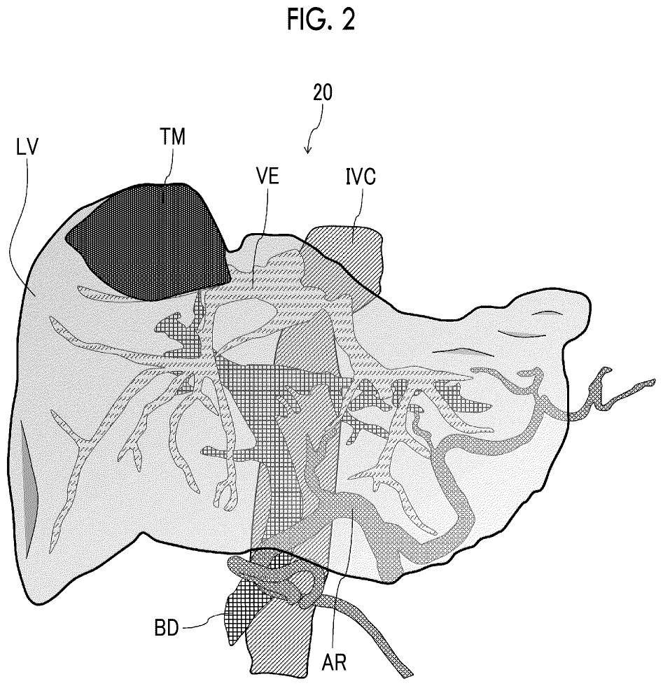

FIG. 2 is a diagram showing an example of a three-dimensional model 20 of the target tissue. In the example of FIG. 2, the three-dimensional model 20 is formed from the medical volume data that includes a liver, an inferior vena cava, a vein, an artery, a bile duct, a tumor, and the like, and the three-dimensional model 20 includes a liver model LV, an inferior vena cava model IVC, a vein model VE, an artery model AR, a bile duct model BD, and a tumor model TM. Normally, the three-dimensional model 20 representing the periphery of the liver includes models of a portal vein, a vascular region, and other tissues, but these are not shown in the three-dimensional model 20.

Since a known method can be used as a method of forming the three-dimensional model 20 based on the medical volume data, detailed description thereof will be omitted here, but the medical image analysis server 14 forms the three-dimensional model 20 using a technique such as volume rendering or surface rendering.

Since the medical volume data has the position information of each voxel, and the three-dimensional model 20 is formed from the medical volume data, the three-dimensional model 20 also has the position information (coordinates) indicating each position. The position information included in the three-dimensional model 20 is information indicating a position in a model space (which is the same space as the data space of the medical volume data).

In addition, the three-dimensional model 20 may have attribute information regarding the target tissue. For example, the three-dimensional model 20 has pieces of information such as a name, a volume, and a diameter of the target tissue in association with the model of each target tissue. These pieces of information may be set through automatic calculations in a case where the medical image analysis server 14 forms the three-dimensional model 20, or may be manually set by an operator of the medical image analysis server 14 or the like.

As mentioned above, since the medical volume data represents the target tissue at a point in time before the real-time ultrasound tomographic image is formed by the ultrasound diagnostic apparatus 16, the three-dimensional model 20 also represents the target tissue at a point in time before the real-time ultrasound tomographic image is formed by the ultrasound diagnostic apparatus 16.

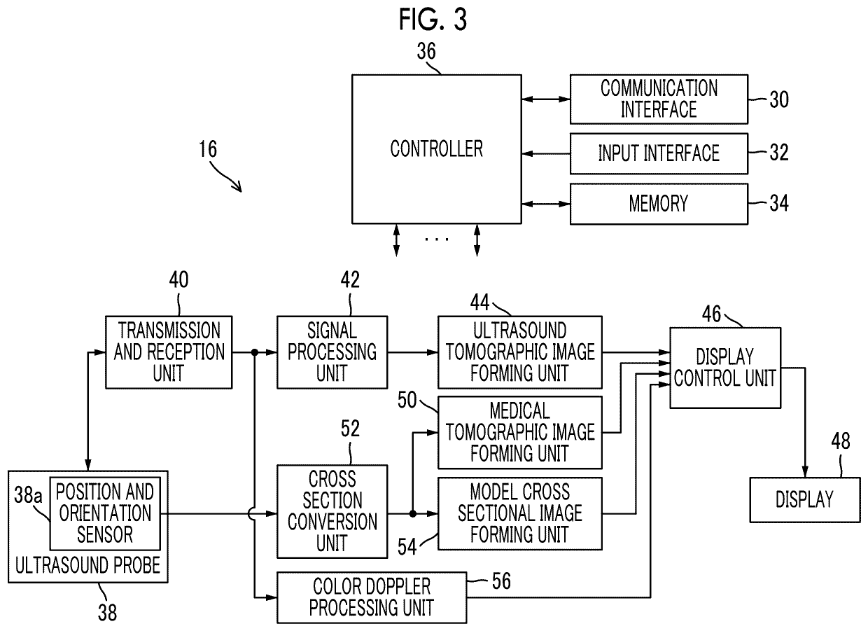

FIG. 3 is a schematic diagram of a configuration of the ultrasound diagnostic apparatus 16 as the ultrasound diagnosis support apparatus according to the present embodiment. The ultrasound diagnostic apparatus 16 is a medical apparatus installed in medical institutions such as a hospital.

A transmission and reception unit 40, a signal processing unit 42, an ultrasound tomographic image forming unit 44, a display control unit 46, a medical tomographic image forming unit 50, a cross section conversion unit 52, a model cross sectional image forming unit 54, and a color Doppler processing unit 56, which are provided in the ultrasound diagnostic apparatus 16, are configured using a processor. The processor includes at least one of a general-purpose processing device (such as a central processing unit (CPU), for example) or a dedicated processing device (such as a graphics processing unit (GPU), an application-specific integrated circuit (ASIC), a field-programmable gate array (FPGA), or a programmable logic device, for example). The processor need not be configured using a single processing device but rather may be configured through the collaboration of a plurality of processing devices that are present at physically separated positions. Additionally, each of the above-described units may be implemented through the collaboration of hardware, such as a processor, and software.

A communication interface 30 is configured using, for example, a network adapter or the like. The communication interface 30 exhibits a function of communicating with other devices via the communication line 18. In particular, in the present embodiment, the communication interface 30 receives the medical volume data of the subject and the three-dimensional model 20 of the target tissue formed based on the medical volume data, from the medical image analysis server 14. In this manner, in the present embodiment, the communication interface 30 exhibits a function as a three-dimensional model acquisition unit.

An input interface 32 is configured using, for example, a button, a trackball, a touch panel, and the like. The input interface 32 is used to input a command of an examiner (such as a doctor or an ultrasound technician) who uses the ultrasound diagnostic apparatus 16 to the ultrasound diagnostic apparatus 16.

A memory 34 includes a hard disk drive (HDD), a solid-state drive (SSD), an embedded MultiMediaCard (eMMC), a read-only memory (ROM), a random-access memory (RAM), or the like. An ultrasound diagnosis support program is stored in the memory 34 in order to operate each unit of the ultrasound diagnostic apparatus 16. The ultrasound diagnosis support program can also be stored in, for example, a computer-readable non-transitory storage medium such as a Universal Serial Bus (USB) memory or a CD-ROM. The ultrasound diagnostic apparatus 16 can read the ultrasound diagnosis support program from such a storage medium and execute the ultrasound diagnosis support program.

A controller 36 includes at least one of a general-purpose processor (such as a CPU, for example) or a dedicated processor (such as a GPU, an ASIC, an FPGA, or a programmable logic device, for example). The controller 36 need not be configured using a single processing device but rather may be configured through the collaboration of a plurality of processing devices that are present at physically separated positions. The controller 36 controls each unit of the ultrasound diagnostic apparatus 16 in accordance with the ultrasound diagnosis support program stored in the memory 34.

An ultrasound probe 38 is a device that transmits and receives ultrasonic waves to and from the subject (the subject related to the three-dimensional model 20 received by the communication interface 30). The ultrasound probe 38 includes a transducer element array consisting of a plurality of transducer elements that transmit and receive ultrasonic waves to and from the subject. In the present embodiment, the transducer element array is formed of a plurality of transducer elements arranged in a single row. The ultrasound probe 38 performs ultrasound scanning in an ultrasound scanning plane that is parallel to an arrangement direction of the plurality of transducer elements and that includes the target tissue.

In the present embodiment, the ultrasound probe 38 includes a position and orientation sensor 38a. The position and orientation sensor 38a is a sensor that detects a current position and orientation of the ultrasound probe 38 and that acquires position and orientation information indicating the current position and orientation of the ultrasound probe 38. That is, the position and orientation sensor 38a exhibits a function as a position and orientation information acquisition unit.

In the present embodiment, the position and orientation sensor 38a is configured using a magnetic sensor, but the position and orientation sensor 38a is not limited thereto. For example, the position and orientation sensor 38a may be an acceleration sensor. In a case where the position and orientation sensor 38a is a magnetic sensor or an acceleration sensor, the position and orientation information acquired by the position and orientation sensor 38a is information indicating the position and orientation in a real space.

In addition, the position and orientation sensor 38a may be a camera provided separately from the ultrasound probe 38. In this case, for example, an AR marker may be attached to the ultrasound probe 38, and the AR marker may be imaged using the camera as the position and orientation sensor 38a, thereby detecting the position and orientation of the ultrasound probe 38. In a case where the position and orientation sensor 38a is a camera, the position and orientation information acquired by the position and orientation sensor 38a is information indicating the position and orientation in a camera coordinate system.

The ultrasound scanning plane in which the ultrasound scanning is performed is determined based on the position and orientation of the ultrasound probe 38 and ultrasonic wave transmission conditions (a scanning method, a transmission output, and the like). Since the ultrasonic wave transmission conditions are determined based on the structure of the ultrasound probe 38, the settings by the examiner, and the like and are known to the ultrasound diagnostic apparatus 16, the position and orientation information of the ultrasound probe 38 is information representing the ultrasound scanning plane.

The transmission and reception unit 40 transmits a transmission signal to the ultrasound probe 38 (specifically, each transducer element of the transducer element array) under the control of the controller 36. As a result, an ultrasonic wave is transmitted from each transducer element toward the subject.

Additionally, the transmission and reception unit 40 receives a reception signal from each transducer element that has received a reflected wave from the subject. The transmission and reception unit 40 includes an adder and a plurality of delay devices corresponding to each transducer element and performs phase alignment and addition processing of aligning and adding phases of the reception signals from the transducer elements by using the adder and the plurality of delay devices. As a result, a reception beam signal is formed in which information indicating the signal intensity of reflected waves from the subject is arranged in a depth direction of the subject.

The signal processing unit 42 executes various types of signal processing including filter processing of applying a bandpass filter, detection processing, and the like, on the reception beam signal from the transmission and reception unit 40.

The ultrasound tomographic image forming unit 44 forms an ultrasound tomographic image (B-mode image) representing an ultrasonic wave transmission and reception plane based on the reception beam signal that has been subjected to signal processing by the signal processing unit 42.

The display control unit 46 performs control to display various images including the ultrasound tomographic image formed by the ultrasound tomographic image forming unit 44 on a display 48. Details of the information displayed on the display 48 by the display control unit 46 will be described below.

The display 48 as a display unit is, for example, a display device configured using a liquid-crystal display, an organic electro luminescence (EL), or the like.

The medical tomographic image forming unit 50 forms a tomographic image by cutting out and reconstructing the medical volume data received from the medical image analysis server 14 along a cross section determined as described below. In the present specification, the tomographic image formed based on the medical volume data is referred to as a medical tomographic image.

The cross section conversion unit 52 converts the ultrasonic wave transmission and reception plane in the real space into a cross section in the model space of the three-dimensional model 20 (as mentioned above, the model space is the same space as the data space of the medical volume data) based on the position and orientation information of the ultrasound probe 38 acquired by the position and orientation sensor 38a. In the present specification, the converted cross section is referred to as a model space cross section.

Various methods can be employed as a method of converting the ultrasonic wave transmission and reception plane into the model space cross section, but in the present embodiment, the conversion is performed as follows.

First, the ultrasound tomographic image forming unit 44 forms the ultrasound tomographic image (referred to as a real-time ultrasound tomographic image in the present specification) based on the reception signal obtained by transmitting and receiving an ultrasonic wave to and from the subject at the current position and orientation of the ultrasound probe 38. In addition, the medical tomographic image forming unit 50 forms the medical tomographic image by cutting out and reconstructing the medical volume data along a predetermined cross section. The predetermined cross section may be determined by being designated by, for example, the examiner.

The display control unit 46 displays the real-time ultrasound tomographic image and the medical tomographic image side by side on the display 48. The examiner adjusts the position and orientation of the ultrasound probe 38 such that the real-time ultrasound tomographic image and the medical tomographic image have exactly the same cross section. Then, in a case where the real-time ultrasound tomographic image and the medical tomographic image have exactly the same cross section, the examiner inputs a calibration instruction using the input interface 32.

The cross section conversion unit 52 stores, in the memory 34, the predetermined cross section set in the medical volume data and the position and orientation information acquired from the position and orientation sensor 38a in a case where the calibration instruction is input, in association with each other. As mentioned above, the ultrasonic wave transmission and reception plane is determined based on the position and orientation information from the position and orientation sensor 38a, thereby defining a correspondence relationship between the cross section (model space cross section) in the model space and the ultrasound scanning plane in the real space (in other words, calibration is performed). Hereinafter, even in a case where the position or the orientation of the ultrasound probe 38 is changed, the cross section conversion unit 52 can specify the model space cross section corresponding to the current ultrasound scanning plane in the model space based on the position and orientation information from the position and orientation sensor 38a. That is, the ultrasound scanning plane can be converted into the model space cross section.

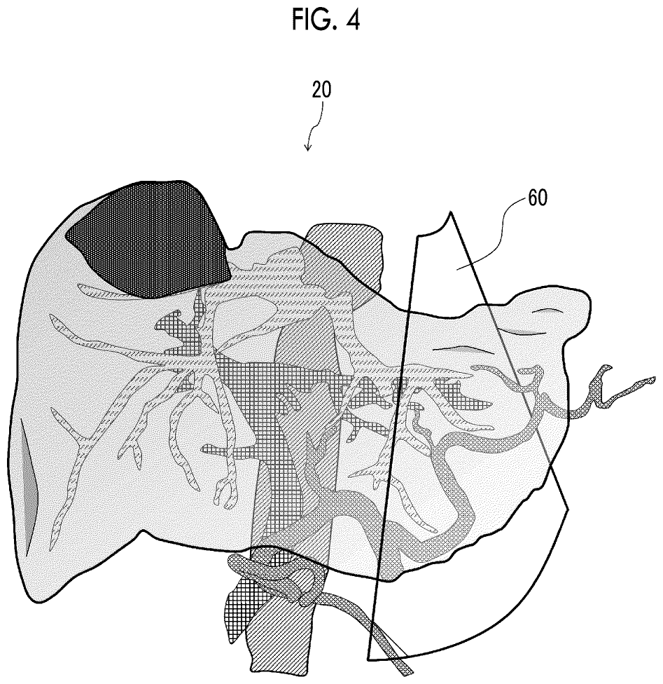

FIG. 4 is a diagram showing an example of a model space cross section 60. The model space cross section 60 may have a shape based on the ultrasonic wave transmission conditions. That is, the cross section conversion unit 52 may determine the shape of the model space cross section 60 based on the ultrasonic wave transmission conditions. For example, in a case where the scanning method of the ultrasound probe 38 is sector scanning, the model space cross section 60 may have a shape similar to that of the ultrasound scanning plane during sector scanning.

The model cross sectional image forming unit 54 forms a cross sectional image by cutting out the three-dimensional model 20 received from the medical image analysis server 14 along the model space cross section 60 obtained by the cross section conversion unit 52. In the present specification, the cross sectional image formed in this manner is referred to as a model cross sectional image.

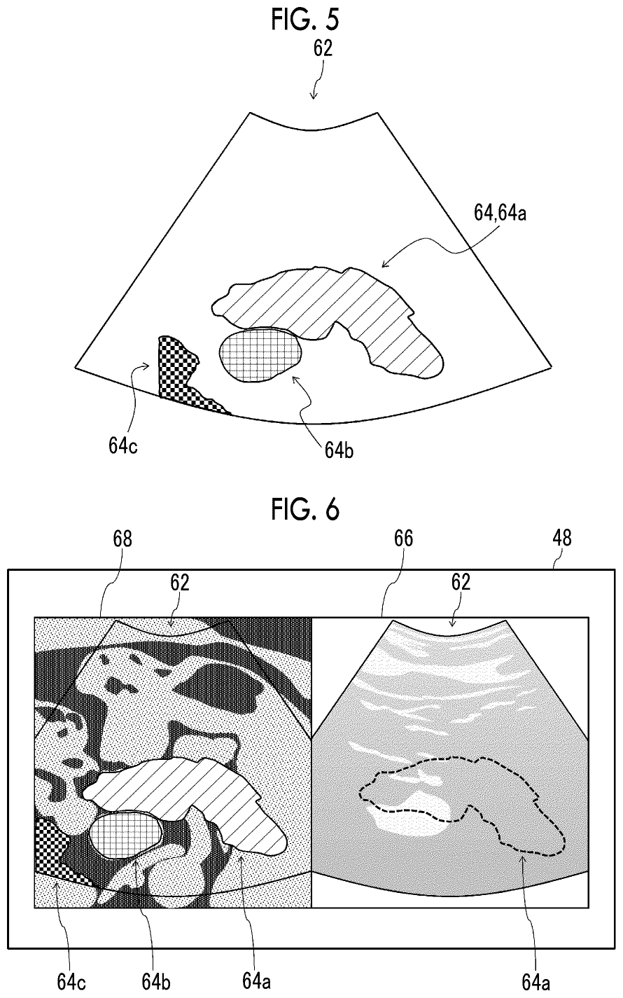

FIG. 5 is a diagram showing an example of a model cross sectional image 62. As mentioned above, since the three-dimensional model 20 represents one or a plurality of target tissues (refer to FIG. 2), the model cross sectional image 62 shows a cross section of each target tissue. In the present specification, a region occupied by the target tissue in the model space cross section 60 is referred to as a target tissue region 64. In the example of FIG. 5, as the target tissue region 64, a target tissue region 64a corresponding to the pancreas, a target tissue region 64b corresponding to the portal vein, and a target tissue region 64c corresponding to the liver are shown.

In other words, the model cross sectional image 62 shows the target tissue region 64 in the model space cross section 60. That is, the model cross sectional image forming unit 54 specifies the target tissue region 64 in the model space cross section 60. Additionally, as mentioned above, since the model space cross section 60 is a cross section corresponding to the current ultrasound scanning plane, it can be said that the model cross sectional image forming unit 54 specifies the target tissue region 64 in the ultrasound scanning plane. In this manner, the model cross sectional image forming unit 54 exhibits a function as a target tissue region specifying unit.

In the present embodiment, since the three-dimensional model 20 has the attribute information regarding each target tissue, the model cross sectional image forming unit 54 can also specify the attribute information (the name, the volume, the diameter, and the like) of the target tissue corresponding to the specified target tissue region 64.

The display control unit 46 displays the real-time ultrasound tomographic image on the display 48 and displays the target tissue region 64 in a superimposed manner on the real-time ultrasound tomographic image. FIG. 6 is a diagram showing a first display example of a real-time ultrasound tomographic image 66 and the target tissue region 64. In the present embodiment, the display control unit 46 performs registration between the real-time ultrasound tomographic image 66 and the model cross sectional image 62 and then superimposes and displays the model cross sectional image 62 on the real-time ultrasound tomographic image 66, thereby displaying the target tissue region 64 in a superimposed manner on the real-time ultrasound tomographic image 66. In the example of FIG. 6, the model cross sectional image 62 showing the target tissue region 64a is superimposed on the real-time ultrasound tomographic image 66. In the example of FIG. 6, the display control unit 46 displays the contour of the target tissue region 64a with a dashed line.

By displaying the target tissue region 64 in a superimposed manner on the real-time ultrasound tomographic image 66, the examiner can easily understand the position of the target tissue to be depicted in the real-time ultrasound tomographic image 66. As a result, the depiction of the target tissue in the real-time ultrasound tomographic image 66 is supported, and the examiner can depict the target tissue in the real-time ultrasound tomographic image 66 quickly and appropriately.

In addition, as mentioned above, since the three-dimensional model 20 represents the target tissue at a (past) point in time before the current point in time, the target tissue region 64 also represents the target tissue in the past. Therefore, by comparing the target tissue depicted in the real-time ultrasound tomographic image 66 with the target tissue region 64, follow-up observation of the subject (target tissue) is possible. For example, in a case where the target tissue is a tumor, by comparing the target tissue region 64 representing the tumor before treatment with the target tissue depicted in the real-time ultrasound tomographic image 66 representing the current tumor, treatment effectiveness determination of the tumor and the like can be performed.

In the example of FIG. 6, the display control unit 46 displays a medical tomographic image 68 and the real-time ultrasound tomographic image 66 side by side. However, the medical tomographic image 68 does not necessarily need to be displayed. The medical tomographic image 68 is formed by the medical tomographic image forming unit 50 cutting out and reconstructing the medical volume data along the model space cross section 60 (as mentioned above, the model space is the same space as the data space of the medical volume data). That is, in a case where the examiner changes the position or the orientation of the ultrasound probe 38, the real-time ultrasound tomographic image 66 naturally changes because the ultrasound scanning plane changes, but since the model space cross section 60 also changes with the change in the ultrasound scanning plane, the medical tomographic image 68 also changes. The real-time ultrasound tomographic image 66 and the medical tomographic image 68 change in synchronization with each other to always represent the same cross section. Consequently, the examiner can perform ultrasound diagnosis while comparing the real-time ultrasound tomographic image 66 with the medical tomographic image 68. As mentioned above, since the medical volume data represents the subject (specifically, the target tissue) at a (past) point in time before the current point in time, the medical tomographic image 68 is an image representing the subject in the past.

In the present embodiment, the display control unit 46 also displays the target tissue region 64 in a superimposed manner on the medical tomographic image 68. In the present embodiment, the display control unit 46 performs registration between the medical tomographic image 68 and the model cross sectional image 62 and then superimposes and displays the model cross sectional image 62 on the medical tomographic image 68, thereby displaying the target tissue region 64 in a superimposed manner on the medical tomographic image 68. In the example of FIG. 6, the model cross sectional image 62 showing the target tissue regions 64a, 64b, and 64c is superimposed on the medical tomographic image 68.

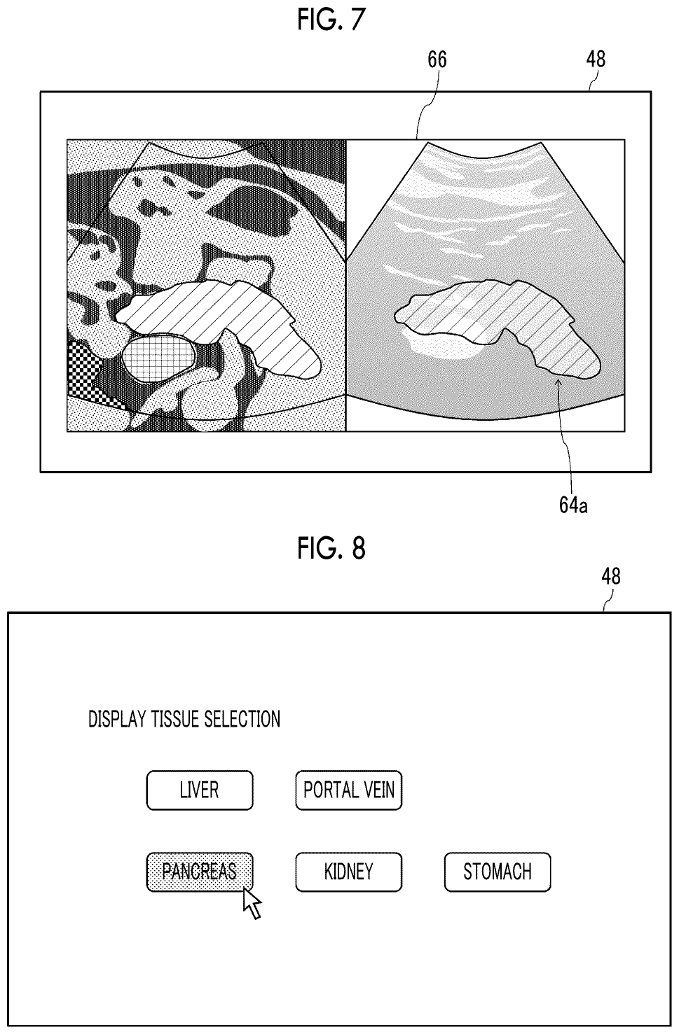

The display control unit 46 may change a display aspect of the target tissue region 64 superimposed on the real-time ultrasound tomographic image 66 in response to an instruction from the examiner. For example, in the example of FIG. 6, the contour of the target tissue region 64a is displayed as a dashed line, but the display control unit 46 may display the target tissue region 64a in a filled manner as shown in FIG. 7 in response to an instruction from the examiner. In a case where the target tissue region 64 is displayed in a filled manner, the display control unit 46 may display the target tissue region 64 in a semi-transparent manner to prevent the target tissue region 64 from obscuring the real-time ultrasound tomographic image 66.

Additionally, in a case where there are a plurality of target tissues and the model cross sectional image forming unit 54 specifies a plurality of target tissue regions 64 as shown in FIG. 5 (in other words, in a case where the model cross sectional image 62 includes a plurality of target tissue regions 64), the display control unit 46 may display the target tissue region 64 selected by the examiner and may not display the target tissue region 64 not selected by the examiner.

For example, the display control unit 46 displays a display tissue selection screen as shown in FIG. 8 on the display 48. On the display tissue selection screen, a list of names of target tissues corresponding to the target tissue regions 64 specified by the model cross sectional image forming unit 54 is displayed. The examiner can select the target tissue on the display tissue selection screen. The display control unit 46 superimposes and displays the target tissue region 64 corresponding to the target tissue selected on the display tissue selection screen on the real-time ultrasound tomographic image 66 and does not display the target tissue region 64 corresponding to the target tissue not selected on the display tissue selection screen.

As a result, for example, in a case where the examiner wants to understand the positional relationship between a plurality of tissues, the target tissue regions 64 can be displayed according to the purpose, such as displaying the plurality of target tissue regions 64, or in a case where tumor treatment is performed, displaying only the target tissue regions 64 corresponding to the tumor and the blood vessel.

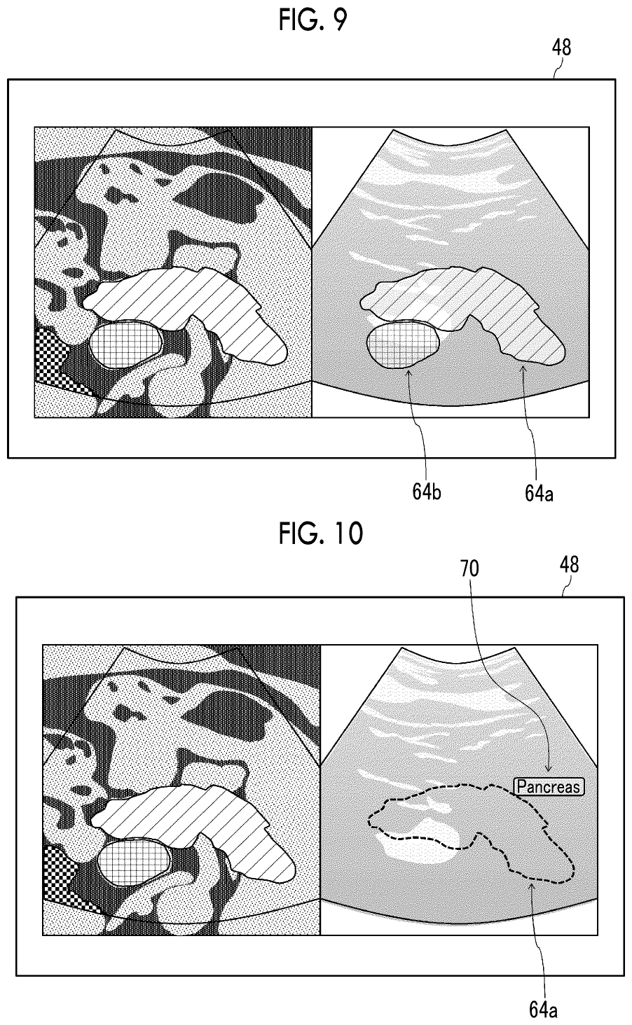

In addition, in a case where there are a plurality of target tissues and the model cross sectional image forming unit 54 specifies a plurality of target tissue regions 64, the display control unit 46 may display each target tissue region 64 in a different aspect. For example, in the example of FIG. 9, the target tissue region 64a and the target tissue region 64b are displayed in different aspects. As a result, the examiner can easily distinguish between the plurality of target tissue regions 64.

Further, the display control unit 46 may display the attribute information of the target tissue corresponding to the target tissue region 64 on the display 48 in an aspect associated with the target tissue region 64. FIG. 10 is a diagram showing a display example of the attribute information of the target tissue. As mentioned above, since the model cross sectional image forming unit 54 can specify the attributes of the target tissue corresponding to the specified target tissue region 64 based on the three-dimensional model 20, the display control unit 46 displays the attribute information specified by the model cross sectional image forming unit 54 on the display 48. In the example of FIG. 10, the name (pancreas) of the target tissue is displayed as attribute information 70 of the target tissue corresponding to the target tissue region 64a in the vicinity of the target tissue region 64a corresponding to the pancreas. Of course, the attribute information 70 to be displayed is not limited to the name of the target tissue and may be the volume and the diameter of the target tissue, or other information. Additionally, the method of associating the target tissue region 64 with the attribute information 70 may be methods such as displaying the target tissue region 64 and the attribute information 70 in the same color, in addition to displaying the attribute information 70 in the vicinity of the target tissue region 64, for example.

By displaying the name of the target tissue as the attribute information 70, it can be used by the examiner for studying anatomy. Additionally, in a case where there are a plurality of target tissue regions 64 corresponding to tumors, cysts, and the like, the examiner can easily understand which target tissue region 64 corresponds to which tumor or cyst. Further, since the attribute information 70 represents the information on the target tissue in the past, for example, by displaying the volume or the diameter of the tumor as the attribute information 70, the examiner can perform the follow-up observation, the treatment diagnosis, or the treatment effectiveness determination through comparison with the tumor depicted in the real-time ultrasound tomographic image 66.

The target tissue region 64 specified by the model cross sectional image forming unit 54 can also be used in the calibration by the cross section conversion unit 52. Specifically, first, the model cross sectional image forming unit 54 forms the model cross sectional image 62 by cutting out the three-dimensional model 20 along the predetermined cross section, thereby specifying the target tissue region 64 in the predetermined cross section, before the conversion processing is performed by the cross section conversion unit 52. In the present specification, the target tissue region 64 is referred to as a registration target tissue region.

Then, the display control unit 46 displays the real-time ultrasound tomographic image 66 and the medical tomographic image 68 side by side on the display 48 and displays the registration target tissue region in a superimposed manner on the real-time ultrasound tomographic image 66, before the conversion processing (calibration) is performed by the cross section conversion unit 52. In the present embodiment, the display control unit 46 performs registration between the real-time ultrasound tomographic image 66 and the model cross sectional image 62 including the registration target tissue region and then superimposes and displays the model cross sectional image 62 on the real-time ultrasound tomographic image 66.

Consequently, the examiner can adjust the position and orientation of the ultrasound probe 38 such that the real-time ultrasound tomographic image 66 and the medical tomographic image 68 have exactly the same cross section by performing registration between the target tissue depicted in the real-time ultrasound tomographic image 66 and the registration target tissue region. That is, it is possible to support the examiner during the calibration and to achieve effects such as improving the accuracy of calibration.

Description returns to FIG. 3. The color Doppler processing unit 56 obtains a Doppler signal by performing quadrature detection processing, autocorrelation calculation, or the like on the reception beam signal formed by the transmission and reception unit 40. The Doppler signal is a signal including information indicating a difference between a transmission frequency and a reception frequency of the ultrasonic wave. The motion velocity of the tissue of the subject can be obtained from the difference between the transmission frequency and the reception frequency based on the Doppler effect, and the Doppler signal can be said to be a signal indicating the motion velocity of the subject tissue.

In particular, the color Doppler processing unit 56 executes the calculation to obtain the Doppler signal in a predetermined region of interest. The region of interest is specified, for example, by the examiner designating a region on the ultrasound tomographic image displayed on the display 48.

Further, the color Doppler processing unit 56 processes the Doppler signal to form a color Doppler image in which the motion velocity of the tissue (for example, the blood flow) of the subject is represented by a color in the region of interest. For example, the color Doppler processing unit 56 forms the color Doppler image in which a portion moving in a direction approaching the ultrasound probe 38 is displayed in red, a portion moving in a direction away from the ultrasound probe 38 is displayed in blue, and the motion velocity is displayed with brightness. A velocity range, which is a range of the motion velocity that can be represented by brightness in the color Doppler image, may be determined by the examiner or the like. The color Doppler image is displayed by being superimposed on the real-time ultrasound tomographic image 66.

In the present embodiment, the color Doppler processing unit 56 may form the color Doppler image based on the target tissue region 64 specified by the model cross sectional image forming unit 54.

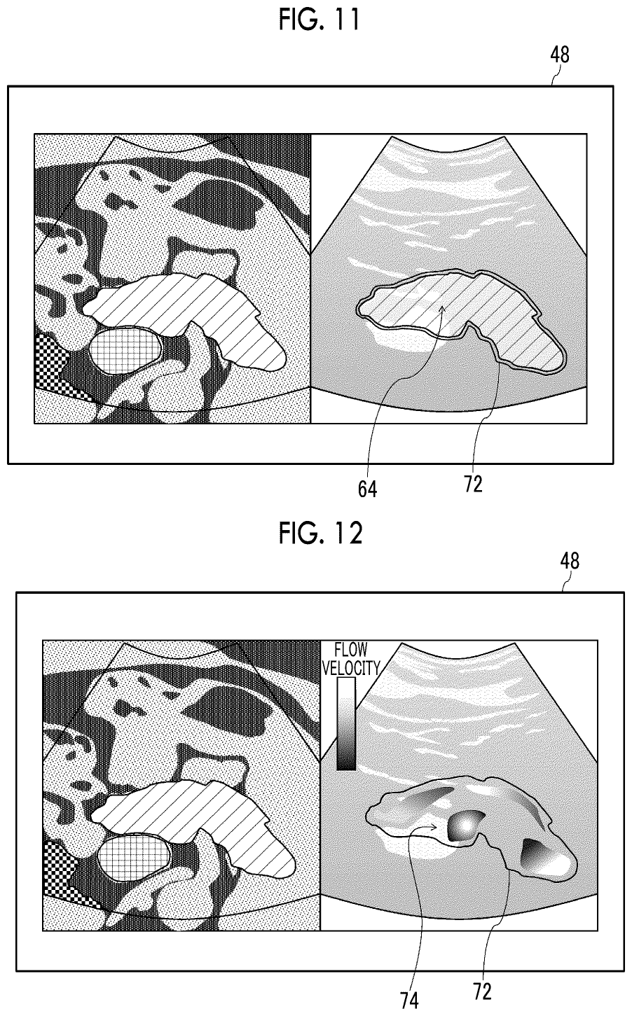

For example, the color Doppler processing unit 56 may set a color Doppler region of interest based on the target tissue region 64. FIG. 11 is a diagram showing an example of a color Doppler region of interest 72 set based on the target tissue region 64. For example, as shown in FIG. 11, the color Doppler processing unit 56 sets a region that is the same as the target tissue region 64 or a region that is slightly larger than the target tissue region 64 and that includes the entire target tissue region 64, as the region of interest 72. As a result, even an examiner who is unfamiliar with setting the color Doppler region of interest 72 can set an appropriate region of interest 72 including the target tissue and can appropriately depict a color Doppler image 74 for a target region, as shown in FIG. 12.

In addition, the color Doppler processing unit 56 may set the color Doppler velocity range based on the target tissue (which is specified by the model cross sectional image forming unit 54 based on the attribute information of the three-dimensional model 20 as mentioned above) corresponding to the target tissue region 64. For example, in a case where the target tissue corresponding to the target tissue region 64 is a tumor, the blood flow flowing through the tumor is slow. Therefore, the color Doppler processing unit 56 sets the velocity range of the color Doppler image related to the target tissue to be lower. On the other hand, in a case where the target tissue corresponding to the target tissue region 64 is an organ, the blood flow flowing through the organ is at least faster than that through the tumor. Therefore, the color Doppler processing unit 56 sets the velocity range of the color Doppler image related to the target tissue to be higher (at least higher than that in a case of the tumor).

The color Doppler processing unit 56 may set the color Doppler region of interest 72 based on the target tissue region 64 and may set the color Doppler velocity range based on the target tissue region 64.

Additionally, the ultrasound tomographic image forming unit 44 may adjust the image quality of the ultrasound tomographic image based on the target tissue region 64 specified by the model cross sectional image forming unit 54. For example, in a case where the target tissue corresponding to the target tissue region 64 is the pancreas, the ultrasound tomographic image forming unit 44 may adjust the ultrasound tomographic image to a brightness suitable for depicting the ultrasound tomographic image of the pancreas. In this case, the ultrasound tomographic image forming unit 44 functions as an image quality adjustment unit. Further, the adjustment of the image quality of the ultrasound tomographic image based on the target tissue region 64 may be performed by the signal processing unit 42 through correction of data before image formation (for example, reception beam data). In this case, the signal processing unit 42 functions as the image quality adjustment unit.

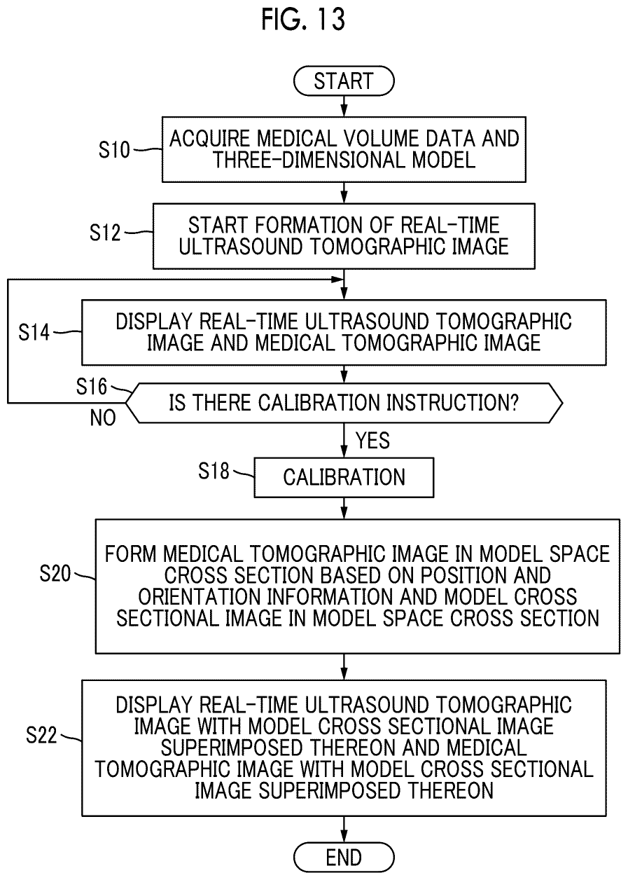

The outline of the configuration of the ultrasound diagnostic apparatus 16 according to the present embodiment is as described above. Hereinafter, a flow of the processing of the ultrasound diagnostic apparatus 16 will be described with reference to a flowchart shown in FIG. 13.

In step S10, the communication interface 30 receives the medical volume data of the subject and the three-dimensional model 20 of the target tissue formed based on the medical volume data, from the medical image analysis server 14.

In step S12, the ultrasound tomographic image forming unit 44 starts the formation of the real-time ultrasound tomographic image 66 based on the reception signal obtained by transmitting and receiving the ultrasonic wave to and from the subject at the current position and orientation of the ultrasound probe 38.

In step S14, the display control unit 46 displays the real-time ultrasound tomographic image 66 formed by the ultrasound tomographic image forming unit 44 and the medical tomographic image 68 formed by the medical tomographic image forming unit 50 cutting out and reconstructing the medical volume data along the predetermined cross section, on the display 48.

In step S16, the examiner adjusts the position and orientation of the ultrasound probe 38 such that the real-time ultrasound tomographic image 66 and the medical tomographic image 68 have exactly the same cross section, and the examiner inputs the calibration instruction using the input interface 32 in a case where the real-time ultrasound tomographic image 66 and the medical tomographic image 68 have exactly the same cross section. The ultrasound tomographic image forming unit 44 repeats the formation of the real-time ultrasound tomographic image 66 for the ultrasound scanning plane based on the position and orientation of the ultrasound probe 38 until the calibration instruction is input. The cross section conversion unit 52 proceeds to step S18 in a case where the calibration instruction is input.

In step S18, the cross section conversion unit 52 stores, in the memory 34, the predetermined cross section set in the medical volume data and the position and orientation information acquired from the position and orientation sensor 38a in a case where the calibration instruction is input, in association with each other. As a result, calibration is performed.

In step S20, the medical tomographic image forming unit 50 forms the medical tomographic image 68 by cutting out and reconstructing the medical volume data acquired in step S10 along the model space cross section 60 specified based on the position and orientation information from the position and orientation sensor 38a. In addition, the model cross sectional image forming unit 54 forms the model cross sectional image 62 by cutting out the three-dimensional model 20 acquired in step S10 along the model space cross section 60.

In step S22, the display control unit 46 performs registration between the real-time ultrasound tomographic image 66 and the model cross sectional image 62 formed in step S20 and then superimposes and displays the model cross sectional image 62 on the real-time ultrasound tomographic image 66. Consequently, the target tissue region 64 is displayed by being superimposed on the real-time ultrasound tomographic image 66. Additionally, the display control unit 46 performs registration between the medical tomographic image 68 formed in step S20 and the model cross sectional image 62 formed in step S20 and then superimposes and displays the model cross sectional image 62 on the medical tomographic image 68.

Although the ultrasound diagnosis support apparatus according to the present disclosure has been described above, the ultrasound diagnosis support apparatus according to the present disclosure is not limited to the above-described embodiment, and various changes can be made without departing from the gist thereof.

For example, in the above-described embodiment, the ultrasound diagnosis support apparatus is the ultrasound diagnostic apparatus 16, but the ultrasound diagnosis support apparatus is not limited to the ultrasound diagnostic apparatus 16. For example, the ultrasound diagnosis support apparatus may be a personal computer (PC), a server, or the like including a processor, a memory, a communication interface, an input interface, and a display. In this case, the processor of the PC or the server as the ultrasound diagnosis support apparatus exhibits functions as the cross section conversion unit 52 and the model cross sectional image forming unit 54, acquires the three-dimensional model 20 from the medical image analysis server 14, acquires the position and orientation information from the position and orientation sensor 38a of the ultrasound probe 38, forms the model cross sectional image 62 based on the acquired three-dimensional model 20 and position and orientation information, and transmits the formed model cross sectional image 62 to the ultrasound diagnostic apparatus, thereby allowing the ultrasound diagnostic apparatus to display the real-time ultrasound tomographic image 66 with the model cross sectional image 62 superimposed thereon.

What is claimed is:

1. An ultrasound diagnosis support apparatus comprising:
a three-dimensional model acquisition unit that acquires a three-dimensional model of a target tissue, which is formed based on medical volume data acquired by a medical device;
a cross section conversion unit that converts an ultrasound scanning plane including the target tissue into a model space cross section, which is a cross section in a model space of the three-dimensional model, based on position and orientation information indicating a current position and orientation of an ultrasound probe that performs ultrasound scanning in the ultrasound scanning plane;
a target tissue region specifying unit that specifies a target tissue region, which is a region occupied by the target tissue in the ultrasound scanning plane, by cutting out the three-dimensional model along the model space cross section; and
a display control unit that displays an ultrasound tomographic image, which is formed based on a reception signal obtained by transmitting and receiving an ultrasonic wave to and from the target tissue at the current position and orientation of the ultrasound probe, on a display unit and that displays the target tissue region in a superimposed manner on the ultrasound tomographic image.

2. The ultrasound diagnosis support apparatus according to claim 1,
wherein the display control unit changes a display aspect of the target tissue region in response to an instruction from an examiner.

3. The ultrasound diagnosis support apparatus according to claim 1,
wherein, in a case where there are a plurality of the target tissues and the target tissue region specifying unit specifies the plurality of target tissue regions, the display control unit displays the target tissue region selected by an examiner and does not display the target tissue region not selected by the examiner.

4. The ultrasound diagnosis support apparatus according to claim 1,
wherein, in a case where there are a plurality of the target tissues and the target tissue region specifying unit specifies the plurality of target tissue regions, the display control unit displays each of the target tissue regions in a different aspect.

5. The ultrasound diagnosis support apparatus according to claim 1,
wherein the three-dimensional model has attribute information regarding the target tissue, and
the display control unit displays the attribute information of the target tissue corresponding to the target tissue region on the display unit in an aspect associated with the target tissue region.

6. The ultrasound diagnosis support apparatus according to claim 1,
wherein the target tissue region specifying unit specifies a registration target tissue region, which is a region occupied by the target tissue in a predetermined cross section, by cutting out the three-dimensional model along the predetermined cross section, and
the display control unit displays, on the display unit, a medical tomographic image, which is formed by cutting out and reconstructing the medical volume data along the predetermined cross section, and the ultrasound tomographic image and further displays the registration target tissue region in a superimposed manner on the ultrasound tomographic image, before conversion processing is performed by the cross section conversion unit.

7. The ultrasound diagnosis support apparatus according to claim 1, further comprising:
a color Doppler processing unit that forms a color Doppler image based on the specified target tissue region.

8. The ultrasound diagnosis support apparatus according to claim 7,
wherein the color Doppler processing unit sets a color Doppler region of interest based on the target tissue region.

9. The ultrasound diagnosis support apparatus according to claim 7,
wherein the color Doppler processing unit sets a color Doppler velocity range based on the target tissue corresponding to the target tissue region.

10. The ultrasound diagnosis support apparatus according to claim 8,
wherein the color Doppler processing unit sets a color Doppler velocity range based on the target tissue corresponding to the target tissue region.

11. The ultrasound diagnosis support apparatus according to claim 5, further comprising:
an image quality adjustment unit that adjusts an image quality of the ultrasound tomographic image based on the target tissue corresponding to the specified target tissue region.

12. A non-transitory computer-readable storage medium storing an ultrasound diagnosis support program for causing a computer to function as:
a three-dimensional model acquisition unit that acquires a three-dimensional model of a target tissue, which is formed based on medical volume data acquired by a medical device;
a cross section conversion unit that converts an ultrasound scanning plane including the target tissue into a model space cross section, which is a cross section in a model space of the three-dimensional model, based on position and orientation information indicating a current position and orientation of an ultrasound probe that performs ultrasound scanning in the ultrasound scanning plane;
a target tissue region specifying unit that specifies a target tissue region, which is a region occupied by the target tissue in the ultrasound scanning plane, by cutting out the three-dimensional model along the model space cross section; and
a display control unit that displays an ultrasound tomographic image, which is formed based on a reception signal obtained by transmitting and receiving an ultrasonic wave to and from the target tissue at the current position and orientation of the ultrasound probe, on a display unit and that displays the target tissue region in a superimposed manner on the ultrasound tomographic image.
